(19) United States
(12) Patent Application Publication
(10) Pub. No.: US 2013/0143239 A1
(43) Pub. Date: Jun. 6, 2013

(54) Melittin Peptide Conjugates And Methods Employing Same

(71) Applicants: David Davoudzadeh, Pleasanton, CA (US); Patrick Bachand, Torrance, CA (US)

(72) Inventors: David Davoudzadeh, Pleasanton, CA (US); Patrick Bachand, Torrance, CA (US)

(73) Assignee: SIEMENS HEALTHCARE DIAGNOSTICS INC., Deerfield, IL (US)

(21) Appl. No.: 13/308,918

(22) Filed: Dec. 1, 2011

(51) Int. Cl. G01N 33/566; C07K 14/435

(52) U.S. Cl. 435/7.92; 530/324; 436/501

(57) ABSTRACT

Methods and reagents are disclosed for conducting assays for IgE specific for honey bee venom allergen. A reagent comprises a conjugate of a small molecule linked to a terminal glycine amino acid of a synthetic 26 amino acid melittin peptide. In the method a combination is provided that comprises a sample and the aforementioned reagent. The combination is subjected to conditions for binding of IgE specific for honey bee venom allergen to the reagent to form a complex. One or both of the presence and amount of the complex is detected and related to one or both of the presence and amount in the sample of IgE specific for honey bee venom allergen.

### BACKGROUND

This invention relates to reagents for use in methods, compositions and kits for determining specific IgE in patients allergic to honey bee venom.

Diagnosis of disease and determination of treatment efficacy are important tools in medicine. In particular, detection of IgE production in an animal can be indicative of disease or other medical condition. Immunoglobulin E (IgE) is the antibody subclass responsible for, among other things, allergic diseases and anaphylactic shock reactions. Measurement of IgE levels in the blood, tissue and body fluids of mammals is generally required for the accurate diagnosis of diseases relating to IgE production. Such diseases include, for example, allergy, atopic disease, hyper IgE syndrome, internal parasite infections and B cell neoplasia. In addition, detection of IgE production in an animal following a treatment involving administration of a medicament is indicative of the efficacy of the treatment, such as when using treatments intended to disrupt IgE production.

Melittin is a main component of bee venom that is responsible for pain in mammals occurring from one or more bee stings. Some people are extremely allergic to bee venom and can experience anaphylactic shock with just one bee sting. Allergy to bee venom is mediated by IgE antibodies that react with components of bee venom. Honey bee venom allergens that are responsible for IgE-mediated allergic reactions include Api m1, Api m2, Api m3, Api m4 and Api m5. Bee venom is also used medically to treat various conditions including some pain-producing diseases and illnesses and to carry out bee venom immunotherapy. Bee venom has been proposed as treatment for chronic injuries (for example, bursitis and tendonitis), hay fever, removal of scar tissue, gout, shingles, burns, fibromyalgia, chronic fatigue syndrome although there is not sufficient evidence as yet to show that bee venom is an effective therapy. There is a continuing need to evaluate subjects for bee venom allergen levels that arise from bee stings and bee venom therapy.

### SUMMARY

Some examples in accordance with the principles described herein are directed to a reagent for determining in a sample the presence and/or amount of an IgE specific for a honey bee venom allergen. The reagent comprises a conjugate of a small molecule linked to the N-terminal glycine amino acid of a synthetic 26 amino acid melittin peptide.

Some examples in accordance with the principles described herein are directed to a method for determining in a sample the presence and/or amount of an IgE specific for a honey bee venom allergen. A combination is provided that comprises a sample and the aforementioned reagent. The combination is subjected to conditions for binding of IgE to the reagent to form a complex. One or both of the presence and amount of the complex is detected and related to one or both of the presence and amount of IgE in the sample.

Some examples in accordance with the principles described herein are directed to a method for determining in a sample one or both of the presence and amount of an IgE specific for Api m4 allergen. A combination is provided that comprises the sample and a reagent comprising a conjugate of biotin and a 26 amino acid melittin peptide wherein the biotin is linked to an amine nitrogen of a terminal glycine of the peptide by means of a linking group comprising repeating ethylene oxide units. The combination is subjected to conditions for binding of IgE to the reagent to form a complex. One or both of the presence and amount of the complex is detected and related to one or both of the presence and amount of IgE in the sample.

### BRIEF DESCRIPTION OF THE DRAWINGS

FIG. 1 is a chemical formula for Biotin-dPEG4-Melittin.

FIG. 2 is a chemical formula Biotin-LC-Melittin.

### DETAILED DESCRIPTION OF SPECIFIC EMBODIMENTS

### General Discussion

Melittin or Api m4 is a 26 amino acid peptide that has the following three-letter code sequence: Gly-Ile-Gly-Ala-Val-Leu-Lys-Val-Leu-Thr-Thr-Gly-Leu-Pro-Ala-Leu-Ile-Ser-Trp-Ile-Lys-Arg-Lys-Arg-Gln-Gln (SEQ ID NO: 1). Some examples in accordance with the principles described herein are directed to conjugates of a small molecule and synthetic, purified melittin. The small molecule is conjugated to the melittin exclusively at the terminal amine nitrogen of melittin, which corresponds to the amine nitrogen of the terminal glycine (Gly) of melittin. Some examples in accordance with the principles described herein are directed to the preparation and use of a purified single-species synthetic biotinylated peptide prepared by solid-phase peptide synthesis as a reagent in the immunodiagnostic detection and/or quantification of specific immunoglobulin E against the allergen Api m4 in sera of patients allergic to honey bee venom.

In some examples, the small molecule has a molecular weight less than about 2000, or less than about 1500, or less than about 1000, or less than about 500, or less than about 400, or less than about 300, for example. Examples of small molecules, by way of illustration and not limitation, include biotin, digoxin, digoxigenin, 2,4-dinitrophenyl, fluorescein, rhodamine, small peptides (meeting the aforementioned molecular weight limits), vitamin B12 and folate, for example. Examples of small molecule-binding partner for the small molecule pairs, by way of illustration and not limitation, include biotin-binding partner for biotin (e.g., avidin, streptavidin and antibody for biotin), digoxin-binding partner for digoxin (e.g., antibody for digoxin), digoxigenin-binding partner for digoxigenin (e.g., antibody for digoxigenin), 2,4-dinitrophenyl and binding partner for 2,4-dinitrophenyl (e.g., antibody for 2,4-dinitrophenyl), fluorescein-binding partner for fluorescein (e.g., antibody for fluorescein), rhodamine-binding partner for rhodamine (e.g., antibody for rhodamine), peptide-binding partner for the peptide (e.g., antibody for the peptide), analyte-specific binding partners (e.g., intrinsic factor for B12, folate binding factor for folate), for example.

As mentioned above, the melittin is synthetically prepared and purified. In some examples, solid phase peptide synthesis is employed to prepare purified single species melittin and conjugates of melittin with a small molecule. Solid-phase peptide synthesis allows the synthesis of small and large peptides. Repeated cycles of protecting with protected amino acid derivatives, coupling, washing, deprotecting and washing are employed. The free N-terminal amine of a peptide attached to a solid-phase is coupled to a single N-protected amino acid unit. This unit is then deprotected, providing a new N-terminal amine to which a further amino acid may be attached. The peptide chains are built using small particulate solid supports referred to as a solid phase. The peptide remains covalently attached to the solid phase until it is cleaved. Immobilization of the peptide on the solid phase allows the solid phase to be manipulated, for example, by washing, to remove unwanted reaction agents and by-products. In this manner, single species, purified melittin is produced.

The solid phase may be, but is not limited to, polymeric supports such as, e.g., polystyrene, polyacrylamide, polyethylene glycol, polypropylene glycol and combinations thereof; glass; cellulose fibers; composites; and resins; for example.

Protecting groups are employed to protect various functional groups of the amino acids so that these functional groups do not react with the reagents for building the amino acid chain. Besides reactive functional groups at the N-terminus and the C-terminus, some amino acids also have side chains that comprise reactive functional groups. These functional groups can react with reactive agents during synthesis such as, for example, reactive agents for adding a small molecule at the N-terminus of the peptide. Thus, in some examples, protecting groups are employed not only during the synthesis of the 26-amino acid melittin peptide but also during the addition of the small molecule at the N-terminal amine group. In some examples, all other reactive functional groups of the synthesized melittin peptide are protected from reaction by means of a protecting group during addition of the small molecule.

The peptide synthesis may involve the use of N-terminal protecting groups, C-terminal protecting groups and side chain protecting groups, for example. The nature of the protecting group is dependent on the nature of the amino acid that is added to the growing chain, the nature of any side chains, the nature of the functional group that is being protected, and the cleavage reagents used at the end of the peptide synthesis, for example. Purified, individual amino acids are reacted with these protecting groups prior to synthesis and then the protecting groups are selectively removed during specific steps of peptide synthesis. N-terminal protecting groups include, but are not limited to, t-butoxycarbonyl (t-Boc), fluorenylmethyloxycarbonyl (Fmoc), acetamidomethyl (Acm), triphenyl methyl (Trt), benzyloxycarbonyl, allyloxycarbonyl (alloc), biphenylisopropyloxycarbonyl, 1-amyloxycarbonyl, isobornyl-oxycarbonyl, alpha-dimethyl-3,5-dimethoxybenzyloxycarbonyl, o-nitrophenylsulfenyl, 2-cyano-1,1-dimentyl-ethoxycarbonyl, bromobenzyloxy, carbamyl, and formyl, for example. The C-terminal carboxyl group is attached to the solid support during the synthesis. The carboxyl group of an amino acid derivative is activated with the use of a peptide coupling agent in each cycle. Peptide coupling agents include O-benzotriazole-N,N,N′,N′-tetramethyl-uronium-hexafluorophosphate (HBTU), O-(N-succinimidyl)-1,1,3,3-tetramethyluronium tetrafluoroborate (TSTU), 2-(1H-7-azabenzotriazol-1-yl)-1,1,3,3-tetramethyluronium hexafluorophosphate (HATU), benzotriazol-1-yl-oxytripyrrolidinophosphonium hexafluorophosphate (PyBOP) and O-(benzotriazol-1-yl)-N,N,N′,N′-tetramethyluronium tetrafluoroborate (TBTU), for example. In some examples, protecting groups for functional groups on side chains of amino acids include, but are not limited to, benzyloxycarbonyl, bromobenzyloxy, dimethoxybenzyloxycarbonyl, tert-butyl, trityl, tosyl and acetamidomethyl, for example. Because peptide synthesis involves a number of different amino acids, protection of functional groups may involve one or more of N-terminal protection, C-terminal protection and side chain protection, thus requiring a number of different protecting groups that are compatible in a particular synthesis.

As mentioned above, the repetitive steps of the peptide synthesis include deprotection of one or more functional groups, i.e., removal of the protecting groups. The nature of the deprotection agent is dependent on the nature of the protecting group, for example. The deprotection agent may be acidic agent such as, for example, trifluoroacetic acid and water, or a basic agent such as, for example, piperidine and DMF. Conditions such as solvents, temperature, pH, and duration of treatment, for example, are dependent on the nature of the protecting group, for example.

The small molecule is activated for reaction with the N-terminal amine nitrogen of the glycine amino acid of the synthesized melittin. Activation may be by way of, but not limited to, a reactive ester such as, for example, N-hydroxysuccinimide (NHS), pentafluorophenyl, or nitrophenyl ester using dicyclohexylcarbodiimide (DCC), diisopropylcarbodiimide (DIC), or 1-ethyl-3-(3-dimethylaminopropyl)carbodiimide (EDC), for example, as condensing agents for activation.

The activated moiety may be bound directly to the small molecule by means of a bond or the activated moiety may be bound to the small molecule through the intermediacy of a linking group. In some examples, the linking group has a molecular weight less than about 2000, or less than about 1500, or less than about 1000, or less than about 500, for example. Such linking groups may comprise about 2 to about 200 atoms, or 4 to about 150 atoms, or about 5 to about 100 atoms, not counting hydrogen and may comprise a chain of from 2 to about 100 atoms, or 3 to about 90 atoms, or about 4 to about 80 atoms, or about 5 to about 70 atoms, or about 10 to about 50 atoms, or about 10 to about 25 atoms, for example, each independently selected from the group consisting of carbon, oxygen, sulfur, nitrogen, and phosphorous. The number of heteroatoms in such linking groups is dependent on the size of the linking group and, in some examples, the number is in the range of from 0 to about 30, or 1 to about 25, or about 2 to about 20, or about 2 to about 15, or about 2 to about 10, or about 3 to about 10, for example. The heteroatoms may be in the form of one or more functionalities, such as, for example, ether, ester, amide, urea, carbamate, sulfonamide, thioether, hydrazone, hydrazide, amidine, and phosphate ester.

In some examples in accordance with the principles disclosed herein, the linking group comprises repeating polyoxyethylene units wherein the number of such units is about 2 to about 10, or about 2 to about 8, or about 2 to about 6, or about 2 to about 4, or about 3 to about 10, or about 3 to about 8, or about 3 to about 6, or about 3 to about 4, for example. In some examples in accordance with the principles disclosed herein, the linking group comprises a hydrocarbon chain wherein the number of carbon atoms in the chain is about 2 to about 40, or about 2 to about 30, or about 2 to about 25, or about 2 to about 20, or about 2 to about 15, or about 2 to about 10, or about 5 to about 40, or about 5 to about 30, or about 5 to about 25, or about 5 to about 20, or about 5 to about 15, or about 5 to about 10, or about 10 to about 40, or about 10 to about 30, or about 10 to about 25, or about 10 to about 20, or about 10 to about 15, for example.

Common functionalities in forming a covalent bond between the linking group and one or both of the small molecule and the melittin peptide are, by way of illustration and not limitation, alkylamine, amidine, thioamide, sulfonamide, ether, ester, urea, thiourea, guanidine, azo, hydrozone, thioether and carboxylate, sulfonate, and phosphate esters, amides and thioesters. For the most part, when a linking group has a linking functionality (functionality for reaction with a moiety) such as, for example, a non-oxocarbonyl group including nitrogen and sulfur analogs, a phosphate group, an amino group, alkylating agent such as halo or tosylalkyl, oxy (hydroxyl or the sulfur analog, mercapto) oxocarbonyl (e.g., aldehyde or ketone), or active olefin such as a vinyl sulfone or α-, β-unsaturated ester, these functionalities are linked to amine groups, carboxyl groups, active olefins, alkylating agents, e.g., bromoacetyl. Where an amine and carboxylic acid, or its nitrogen derivative or phosphoric acid derivative, are linked, amides, amidines and phosphoramides are formed, respectively. Where mercaptan and activated olefin are linked, thioethers are formed. Where a mercaptan and an alkylating agent are linked, thioethers are formed. Where aldehyde and an amine are linked under reducing conditions, an alkylamine is formed. Where a ketone or aldehyde and a hydroxylamine (including derivatives thereof where a substituent is in place of the hydrogen of the hydroxyl group) or hydrazine are linked, an oxime functionality (═N—O—) or hydrazone (═N—NH—) functionality is formed. Where a carboxylic acid or phosphate acid and an alcohol are linked, esters are formed.

As mentioned above, embodiments of the conjugates of a small molecule and the synthetic melittin peptide may be employed in methods for determining the presence and/or amount of an IgE specific for a honey bee venom allergen (i.e., analyte) in a sample. A combination is provided that comprises a sample and the aforementioned reagent. The combination is subjected to conditions for binding of IgE analyte to the reagent to form a complex. One or both of the presence and amount of the complex is detected and related to one or both of the presence and amount of IgE analyte in the sample.

The sample to be analyzed is one that is suspected of containing IgE specific for honey bee venom allergen because the patient may be experiencing a factor that would be responsible for production of such IgE, for example, therapy using honey bee venom or a sting from one or more honey bees. The samples are preferably from a mammalian subject, e.g., humans or other animal species and include biological fluids such as whole blood, serum, plasma, sputum, lymphatic fluid, semen, vaginal mucus, feces, urine, spinal fluid, saliva, stool, cerebral spinal fluid, tears, mucus, and the like; biological tissue such as hair, skin, sections or excised tissues from organs or other body parts; and so forth. In many instances, the sample is whole blood, plasma or serum.

The sample can be prepared in any convenient medium. Conveniently, the sample may be prepared in an assay medium, which is discussed more fully hereinbelow. In some instances a pretreatment may be applied to the sample such as, for example, to lyse blood cells. In some examples, such pretreatment is performed in a medium that does not interfere subsequently with an assay.

The assays are normally carried out in an aqueous buffered medium at a moderate pH, generally that which provides optimum assay sensitivity. The aqueous medium may be solely water or may include from 0.1 to about 40 volume percent of a cosolvent. The pH for the medium will be in the range of about 4 to about 11, or in the range of about 5 to about 10, or in the range of about 6.5 to about 9.5. The pH will usually be a compromise between optimum binding of the binding members of any specific binding pairs, the pH optimum for other reagents of the assay such as members of the signal producing system, and so forth. Various buffers may be used to achieve the desired pH and maintain the pH during the assay. Illustrative buffers include, but are not limited to, borate, phosphate, carbonate, tris, barbital, PIPES, HEPES, MES, ACES, MOPS, BICINE, and TRICINE, for example. The particular buffer employed is not critical, but in an individual assay one or another buffer may be preferred.

Various ancillary materials may be employed in the assay methods. For example, in addition to buffers the medium may comprise stabilizers for the medium and for the reagents employed. In some embodiments, in addition to these additives, proteins may be included, such as albumins; organic solvents such as formamide; quaternary ammonium salts; polyanions such as dextran sulfate; binding enhancers, e.g., polyalkylene glycols; polysaccharides such as dextran, trehalose, or the like. The medium may also comprise agents for preventing the formation of blood clots. Such agents are well known in the art and include, for example, EDTA, EGTA, citrate, heparin, and the like. The medium may also comprise one or more preservatives as are known in the art such as, for example, sodium azide, neomycin sulfate, PROCLIN® 300, Streptomycin, and the like. Any of the above materials, if employed, is present in a concentration or amount sufficient to achieve the desired effect or function.

The sample and a reagent for determining the presence and/or amount of an IgE specific for honey bee venom allergen in a sample are combined in the assay medium. The reagent comprises a conjugate of a small molecule and a synthetic melittin peptide. IgE specific for honey bee venom allergen in the sample, if present, binds to the reagent. Depending on the nature of the assay employed, the reagent that comprises the conjugate may also comprise one or more components such as, for example, a solid support (e.g., a particle) or a member of a signal producing system (e.g., an enzyme (alkaline phosphatase, β-galactosidase, and horseradish peroxidase), biotin, a chemiluminescent or fluorescent label, a sensitizer or a radioisotope), for example. Furthermore, again depending on the nature of the assay employed, other reagents may also be included in the initial combination or added subsequently. Such reagents include additional binding agents such as, for example, one or more antibodies, e.g., antibodies for IgE, and members of a signal producing system, for example.

One or more incubation periods may be applied to the medium at one or more intervals including any intervals between additions of various reagents employed in an assay including those mentioned above. The medium is usually incubated at a temperature and for a time sufficient for binding of various components of the reagents and binding of IgE specific for honey bee venom allergen in the sample to occur. Moderate temperatures are normally employed for carrying out the method and usually constant temperature, preferably, room temperature, during the period of the measurement. In some examples, incubation temperatures range from about 5° to about 99° C., or from about 15° C. to about 70° C., or about 20° C. to about 45° C. The time period for the incubation, in some examples, is about 0.2 seconds to about 24 hours, or about 1 second to about 6 hours, or about 2 seconds to about 1 hour, or about 1 minute to about 15 minutes. The time period depends on the temperature of the medium and the rate of binding of the various reagents, which is determined by the association rate constant, the concentration, the binding constant and dissociation rate constant.

### General Description of Assays for an IgE Specific for Honey Bee Venom Allergen Utilizing the Present Reagents

A conjugate of the small molecule and the synthetic melittin peptide in accordance with the principles described herein may be employed in the determination of IgE specific for honey bee venom allergen using a number of different assay formats. In general, in such assays the reagents comprise, among others, the above conjugate. A sample suspected of containing IgE specific for honey bee venom allergen is combined in an assay medium with the above conjugate. A determination is made of the extent of binding between IgE specific for honey bee venom allergen and the present conjugate reagent. In some examples, a labeled reagent specific for IgE may also be employed in some embodiments for detection of the binding event between IgE specific for honey bee venom allergen and the conjugate reagent. The assay can be performed either without separation (homogeneous) or with separation (heterogeneous) of any of the assay components or products. Heterogeneous assays usually involve one or more separation steps and can be competitive or non-competitive.

Immunoassays may involve labeled or non-labeled reagents. Immunoassays involving non-labeled reagents usually comprise the formation of relatively large complexes involving one or more antibodies. Such assays include, for example, immunoprecipitin and agglutination methods and corresponding light scattering techniques such as, e.g., nephelometry and turbidimetry, for the detection of antibody complexes. Labeled immunoassays include chemiluminescence immunoassays, enzyme immunoassays, fluorescence polarization immunoassays, radioimmunoassay, inhibition assay, induced luminescence, fluorescent oxygen channeling assay, and so forth.

One general group of immunoassays in which the conjugate of a small molecule and synthetic melittin peptide may be employed to determine the presence and/or amount of IgE specific for honey bee venom allergen in a sample includes immunoassays using a limited concentration of the present conjugate reagent. Another group of immunoassays involves the use of an excess of one or more of the principal reagents such as, for example, an excess of the present conjugate reagent. Another group of immunoassays are separation-free homogeneous assays in which the labeled reagents modulate the label signal upon binding of the present conjugate to IgE specific for honey bee venom allergen in the sample.

As mentioned above, the assays can be performed either without separation (homogeneous) or with separation (heterogeneous) of any of the assay components or products. Homogeneous immunoassays are exemplified by the EMIT® assay (Siemens Healthcare Diagnostics Inc., Deerfield, Ill.) disclosed in Rubenstein, et al., U.S. Pat. No. 3,817,837, column 3, line 6 to column 6, line 64; immunofluorescence methods such as those disclosed in Ullman, et al., U.S. Pat. No. 3,996,345, column 17, line 59, to column 23, line 25; enzyme channeling immunoassays (“ECIA”) such as those disclosed in Maggio, et al., U.S. Pat. No. 4,233,402, column 6, line 25 to column 9, line 63; the fluorescence polarization immunoassay (“FPIA”) as disclosed, for example, in, among others, U.S. Pat. No. 5,354,693; and enzyme immunoassays such as the enzyme-linked immunosorbent assay (“ELISA”). Exemplary of heterogeneous assays are the radioimmunoassay, disclosed in Yalow, et al., J. Clin. Invest. 39:1157 (1960). The relevant portions of the above disclosures are all incorporated herein by reference.

Other enzyme immunoassays are the enzyme modulate mediated immunoassay (“EMMIA”) discussed by Ngo and Lenhoff, FEBS Lett. (1980) 116:285-288; the substrate labeled fluorescence immunoassay (“SLFIA”) disclosed by Oellerich, J. Clin. Chem. Clin. Biochem. (1984) 22:895-904; the combined enzyme donor immunoassays (“CEDIA”) disclosed by Khanna, et al., Clin. Chem. Acta (1989) 185:231-240; homogeneous particle labeled immunoassays such as particle enhanced turbidimetric inhibition immunoassays (“PETINIA”), particle enhanced turbidimetric immunoassay (“PETIA”), etc.; and the like.

Other assays include the sol particle immunoassay (“SPIA”), the disperse dye immunoassay (“DIA”); the metalloimmunoassay (“MIA”); the enzyme membrane immunoassays (“EMIA”); luminoimmunoassays (“LIA”); and so forth. Other types of assays include immunosensor assays involving the monitoring of the changes in the optical, acoustic and electrical properties of the present conjugate upon the binding of IgE analyte. Such assays include, for example, optical immunosensor assays, acoustic immunosensor assays, semiconductor immunosensor assays, electrochemical transducer immunosensor assays, potentiometric immunosensor assays, amperometric electrode assays.

Heterogeneous assays usually involve one or more separation steps and can be competitive or non-competitive. A variety of competitive and non-competitive heterogeneous assay formats are disclosed in Davalian, et al., U.S. Pat. No. 5,089,390, column 14, line 25 to column 15, line 9, incorporated herein by reference. In a typical competitive heterogeneous assay, a support having the present conjugate bound thereto is contacted with a medium containing the sample suspected of containing IgE analyte and IgE conjugated to a detectable label such as an enzyme. IgE in the sample competes with the IgE conjugate bearing the detectable label for binding to the present conjugate. After separating the support and the medium, the label activity of the support or the medium is determined by conventional techniques and is related to the amount of IgE analyte in the sample.

The support may be comprised of an organic or inorganic, solid or fluid, water insoluble material, which may be transparent or partially transparent. The support can have any of a number of shapes, such as a particle (particulate support) including bead, a film, a membrane, a tube, a well, a strip, a rod, and planar surfaces such as, e.g., plate, paper, etc., fiber, for example. The support may or may not be suspendable in the medium in which it is employed. Examples of suspendable supports are polymeric materials such as latex, lipid bilayers or liposomes, oil droplets, cells and hydrogels, and magnetic particles, for example. Other support compositions include polymers, such as nitrocellulose, cellulose acetate, poly (vinyl chloride), polyacrylamide, polyacrylate, polyethylene, polypropylene, poly(4-methylbutene), polystyrene, polymethacrylate, poly(ethylene terephthalate), nylon, poly(vinyl butyrate), etc.; either used by themselves or in conjunction with other materials.

In some assay examples the support may be a particle. The particles have an average diameter of at least about 0.02 microns and not more than about 100 microns. In some examples, the particles have an average diameter from about 0.05 microns to about 20 microns, or from about 0.3 microns to about 10 microns. The particle may be organic or inorganic, swellable or non-swellable, porous or non-porous, preferably of a density approximating water, generally from about 0.7 g/mL to about 1.5 g/mL, and composed of material that can be transparent, partially transparent, or opaque. The particles can be biological materials such as cells and microorganisms, e.g., erythrocytes, leukocytes, lymphocytes, hybridomas, streptococcus, Staphylococcus aureus, and E. coli, viruses, for example. The particles can also be particles comprised of organic and inorganic polymers, liposomes, latex particles, magnetic or non-magnetic particles, phospholipid vesicles, chylomicrons, lipoproteins, and the like. In some examples, the particles are chromium dioxide (chrome) particles or latex particles.

In a typical non-competitive sandwich assay, an immune sandwich complex is formed in an assay medium. The complex comprises the IgE analyte, a conjugate of a small molecule and melittin and an antibody that binds to the IgE analyte or a complex of the IgE analyte and the present conjugate reagent. Subsequently, the immune sandwich complex is detected and is related to the amount of IgE analyte in the sample. The immune sandwich complex is detected by virtue of the presence in the complex of a label wherein either or both the present conjugate reagent and the antibody for IgE contain labels or substituents capable of combining with labels. In one approach in a sandwich assay, a first incubation of unlabeled conjugate reagent coupled to a solid support such as a particle is contacted with a medium containing a sample suspected of containing the IgE analyte. After a wash and separation step, the support is contacted with a medium containing an antibody for IgE, which contains a label such as an enzyme and which need only be an antibody specific for an IgE not necessarily specific for the IgE analyte, for a second incubation period. The support is again washed and separated from the medium and either the medium or the support is examined for the presence of label. The presence and amount of label is related to the presence or amount of the IgE analyte.

In a variation of the above sandwich assay, the sample suspected of containing IgE specific for honey bee venom allergen in a suitable medium is contacted with labeled antibody for IgE and incubated for a period of time. Then, the medium is contacted with a support to which is bound a conjugate of a small molecule and synthetic melittin peptide in accordance with the principles described herein. After an incubation period, the support is separated from the medium and washed to remove unbound reagents. The support or the medium is examined for the presence of the label, which is related to the presence or amount of IgE specific for honey bee venom allergen. In another variation of the above, the sample, the present conjugate bound to a support and the labeled antibody are combined in a medium and incubated in a single incubation step. Separation, wash steps and examination for label are as described above.

In many of the assays discussed herein, a label is employed; the label is usually part of a signal producing system (“sps”). The nature of the label is dependent on the particular assay format. An sps usually includes one or more components, at least one component being a detectable label, which generates a detectable signal that relates to the amount of bound and/or unbound label, i.e. the amount of label bound or not bound to the IgE analyte being detected or to an agent that reflects the amount of the IgE analyte to be detected. The label is any molecule that produces or can be induced to produce a signal, and may be, for example, an enzyme, a fluorescer, a chemiluminescer, a photosensitizer, or a radiolabel. Thus, the signal is detected and/or measured by detecting enzyme activity, luminescence, light absorbance or radioactivity, respectively.

Suitable labels include, by way of illustration and not limitation, enzymes such as alkaline phosphatase, glucose-6-phosphate dehydrogenase (“G6PDH”), β-galactosidase, and horseradish peroxidase; ribozyme; a substrate for a replicase such as QB replicase; promoters; dyes; fluorescers, such as fluorescein, isothiocyanate, rhodamine compounds, phycoerythrin, phycocyanin, allophycocyanin, o-phthalaldehyde, and fluorescamine; complexes such as those prepared from CdSe and ZnS present in semiconductor nanocrystals known as Quantum Dots; chemiluminescers such as luminal and isoluminol; sensitizers; coenzymes; enzyme substrates; radiolabels such as ¹²⁵I, ¹³¹I, ¹⁴C, ³H, ⁵⁷Co and ⁷⁵Se; particles such as latex particles, carbon particles, metal particles including magnetic particles, e.g., chromium dioxide (CrO₂) particles, and the like; metal sol; crystallite; liposomes; cells, etc., which may be further labeled with a dye, catalyst or other detectable group. Suitable enzymes and coenzymes are disclosed in Litman, et al., U.S. Pat. No. 4,275,149, columns 19-28, and Boguslaski, et al., U.S. Pat. No. 4,318,980, columns 10-14; suitable fluorescers and chemiluminescers are disclosed in Litman, et al., U.S. Pat. No. 4,275,149, at columns 30 and 31; which are incorporated herein by reference.

The label can directly produce a signal and, therefore, additional components are not required to produce a signal. Numerous organic molecules, for example fluorescers, are able to absorb ultraviolet and visible light, where the light absorption transfers energy to these molecules and elevates them to an excited energy state. This absorbed energy is then dissipated by emission of light at a longer wavelength. Other labels that directly produce a signal include radioactive isotopes and dyes.

Alternately, the label may need other components to produce a signal, and the signal producing system would then include all the components required to produce a measurable signal. Such other components may include substrates, coenzymes, enhancers, additional enzymes, substances that react with enzymatic products, catalysts, activators, cofactors, inhibitors, scavengers, metal ions, and a specific binding substance required for binding of signal generating substances. A detailed discussion of suitable signal producing systems can be found in Ullman, et al., U.S. Pat. No. 5,185,243, columns 11-13, incorporated herein by reference.

In some embodiments the enzymes are redox enzymes, particularly dehydrogenases such as glucose-6-phosphate dehydrogenase, lactate dehydrogenase, etc., and enzymes that involve the production of hydrogen peroxide and the use of the hydrogen peroxide to oxidize a dye precursor to a dye. Particular combinations include saccharide oxidases, e.g., glucose and galactose oxidase, or heterocyclic oxidases, such as uricase and xanthine oxidase, coupled with an enzyme which employs the hydrogen peroxide to oxidize a dye precursor, that is, a peroxidase such as horseradish peroxidase, lactoperoxidase, or microperoxidase. Additional enzyme combinations are known in the art. When a single enzyme is used as a label, other enzymes may find use such as hydrolases, transferases, and oxidoreductases, preferably hydrolases such as alkaline phosphatase and beta-galactosidase. Alternatively, luciferases may be used such as firefly luciferase and bacterial luciferase.

Illustrative co-factors and co-enzymes that find use include NAD[H], NADP[H], pyridoxal phosphate, FAD[H], FMN[H], etc., usually coenzymes involving cycling reactions. See, for example, U.S. Pat. No. 4,318,980, the disclosure of which is incorporated herein by reference.

Some known assays utilize a signal producing system (sps) that employs first and second sps members. The designation “first” and “second” is completely arbitrary and is not meant to suggest any order or ranking among the sps members or any order of addition of the sps members in the present methods. The sps members may be related in that activation of one member of the sps produces a product such as, e.g., light, which results in activation of another member of the sps.

In some embodiments of known assays, the sps members comprise a sensitizer such as, for example, a photosensitizer, and a chemiluminescent composition where activation of the sensitizer results in a product that activates the chemiluminescent composition. The second sps member usually generates a detectable signal that relates to the amount of bound and/or unbound sps member, i.e., the amount of sps member bound or not bound to the IgE analyte being detected or to an agent that reflects the amount of the IgE analyte to be detected. In some examples in accordance with the principles described herein, one of either the sensitizer reagent or the chemiluminescent reagent comprises the present conjugate reagent. Examples of photosensitizers and chemiluminescent reagents that may be utilized are those set forth in U.S. Pat. Nos. 5,340,716 and 6,251,581, the relevant disclosures of which are incorporated herein by reference.

In a particular embodiment, an induced luminescence immunoassay may be employed where the assay utilizes a conjugate of a small molecule and synthetic melittin peptide in accordance with the principles described herein. The induced luminescence immunoassay is referred to in U.S. Pat. No. 5,340,716 (Ullman), which disclosure is incorporated herein by reference. In one approach, the assay uses a particle having associated therewith a photosensitizer where the conjugate in accordance with the principles described herein is bound to the particle. The chemiluminescent reagent comprises a binding partner for IgE, for example, antibody for IgE. The present conjugate binds to the IgE analyte to form a complex, or binds to a second sbp member to form a complex, in relation to the presence of the IgE analyte. If the IgE analyte is present, the photosensitizer and the chemiluminescent compound come into close proximity by virtue of the binding, to the IgE analyte, of the melittin that is part of the small molecule-melittin conjugate in accordance with the principles described herein. The photosensitizer generates singlet oxygen and activates the chemiluminescent reagent when the two labels are in close proximity. The activated chemiluminescent reagent subsequently produces light. The amount of light produced is related to the amount of the complex formed, which in turn is related to the amount of IgE analyte present in the sample.

In some embodiments of the induced luminescence assay, a photosensitizer particle is employed that is conjugated to avidin or streptavidin. The present conjugate comprising biotin linked to the synthetic melittin peptide is also employed. A chemiluminescent reagent that comprises a binding partner for IgE is employed as part of the detection system. The reaction medium is incubated to allow the avidin or streptavidin of the photosensitizer particles to bind to the biotin-synthetic melittin peptide conjugate by virtue of the binding between avidin and biotin and to also allow the binding partner for the IgE analyte that is part of the chemiluminescent reagent to bind to the IgE analyte. Then, the medium is irradiated with light to excite the photosensitizer, which is capable in its excited state of activating oxygen to a singlet state. Because the chemiluminescent reagent is now in close proximity to the photosensitizer by virtue of the presence of the IgE analyte, it is activated by the singlet oxygen and emits luminescence. The medium is then examined for the presence and/or the amount of luminescence or light emitted, the presence thereof being related to the presence and/or amount of the IgE analyte.

The concentration of the IgE analyte that may be assayed generally varies from about 10⁻⁵ to about 10⁻¹⁷ M, more usually from about 10⁻⁶ to about 10⁻¹⁴ M. Considerations, such as whether the assay is qualitative, semi-quantitative or quantitative (relative to the amount of the IgE analyte present in the sample), the particular detection technique and the expected concentration of the IgE analyte normally determine the concentrations of the various reagents.

The concentrations of the various reagents in the assay medium will generally be determined by the concentration range of interest of the IgE analyte, the nature of the assay, and the like. However, the final concentration of each of the reagents is normally determined empirically to optimize the sensitivity of the assay over the range of interest. That is, a variation in concentration of IgE analyte that is of significance should provide an accurately measurable signal difference. Considerations such as the nature of the signal producing system and the nature of the analytes normally determine the concentrations of the various reagents.

As mentioned above, the sample and reagents are provided in combination in the medium. While the order of addition to the medium may be varied, there will be certain preferences for some embodiments of the assay formats described herein. The simplest order of addition, of course, is to add all the materials simultaneously and determine the effect that the assay medium has on the signal as in a homogeneous assay. Alternatively, each of the reagents, or groups of reagents, can be combined sequentially. In some embodiments, an incubation step may be involved subsequent to each addition as discussed above. In heterogeneous assays, washing steps may also be employed after one or more incubation steps.

### Examination Step

In a next step of an assay method, the medium is examined for the presence of a complex comprising the IgE analyte and the conjugate of the small molecule and synthetic melittin peptide. The presence and/or amount of the complex indicates the presence and/or amount of the IgE analyte in the sample.

The phrase “measuring the amount of an IgE analyte” refers to the quantitative, semiquantitative and qualitative determination of the IgE specific for honey bee venom allergen. Methods that are quantitative, semiquantitative and qualitative, as well as all other methods for determining the IgE analyte, are considered to be methods of measuring the amount of the IgE analyte. For example, a method, which merely detects the presence or absence of the IgE analyte in a sample suspected of containing the IgE analyte, is considered to be included within the scope of the present invention. The terms “detecting” and “determining,” as well as other common synonyms for measuring, are contemplated within the scope of the present invention.

In many embodiments the examination of the medium involves detection of a signal from the medium. The presence and/or amount of the signal is related to the presence and/or amount of the IgE analyte in the sample. The particular mode of detection depends on the nature of the sps. As discussed above, there are numerous methods by which a label of an sps can produce a signal detectable by external means. Activation of a signal producing system depends on the nature of the signal producing system members.

Temperatures during measurements generally range from about 10° C. to about 70° C. or from about 20° C. to about 45° C., or about 20° C. to about 25° C. In one approach standard curves are formed using known concentrations of the IgE analyte. Calibrators and other controls may also be used.

Luminescence or light produced from any label can be measured visually, photographically, actinometrically, spectrophotometrically, such as by using a photomultiplier or a photodiode, or by any other convenient means to determine the amount thereof, which is related to the amount of IgE analyte in the medium. The examination for presence and/or amount of the signal also includes the detection of the signal, which is generally merely a step in which the signal is read. The signal is normally read using an instrument, the nature of which depends on the nature of the signal. The instrument may be, but is not limited to, a spectrophotometer, fluorometer, absorption spectrometer, luminometer, and chemiluminometer, for example.

### Kits Comprising Reagents for Conducting Assays

The present conjugate of a small molecule and synthetic melittin peptide and other reagents for conducting a particular assay for the IgE specific for honey bee venom allergen analyte may be present in a kit useful for conveniently performing an assay for the determination of an IgE analyte. In some embodiments a kit comprises in packaged combination a biotin-binding partner such as, for example, avidin or streptavidin, associated with a particle, biotinylated synthetic melittin peptide in accordance with the principles described herein and an enzyme labeled antibody for the IgE analyte. The kit may further include other reagents for performing the assay, the nature of which depend upon the particular assay format.

The reagents may each be in separate containers or various reagents can be combined in one or more containers depending on the cross-reactivity and stability of the reagents. The kit can further include other separately packaged reagents for conducting an assay such as additional specific binding partner (sbp) members, sps members, ancillary reagents, for example.

The relative amounts of the various reagents in the kits can be varied widely to provide for concentrations of the reagents that substantially optimize the reactions that need to occur during the present methods and further to optimize substantially the sensitivity of an assay. Under appropriate circumstances one or more of the reagents in the kit can be provided as a dry powder, usually lyophilized, including excipients, which on dissolution will provide for a reagent solution having the appropriate concentrations for performing a method or assay using a conjugate of a small molecule and melittin in accordance with the principles described herein. The kit can further include a written description of a method utilizing reagents that include a conjugate in accordance with the principles described herein.

The phrase “at least” as used herein means that the number of specified items may be equal to or greater than the number recited. The phrase “about” as used herein means that the number recited may differ by plus or minus 10%; for example, “about 5” means a range of 4.5 to 5.5. The designations “first” and “second” are used solely for the purpose of differentiating between two items such as, for example, “first sps member” and “second sps member,” and are not meant to imply any sequence or order or importance to one item over another.

The following discussion is directed to specific examples in accordance with the principles described herein by way of illustration and not limitation; the specific examples are not intended to limit the scope of the present disclosure and the appended claims. Numerous modifications and alternative compositions, methods, and systems may be devised without departing from the spirit and scope of the present disclosure.

### EXAMPLES

Unless otherwise indicated, materials in the experiments below may be purchased from the Sigma-Aldrich Chemical Corporation, St. Louis Mo. The amino acid derivatives for peptide synthesis may be purchased from EMD Chemicals, Gibbstown N.J. Biotin-dPEG4-NHS ester may be purchased from Quanta Biodesign, Powell Ohio. Parts and percentages disclosed herein are by weight unless otherwise indicated.

Definitions: MUXF allergen=IgE binding oligosaccharide; DMF=dimethylformamide; HPLC=high performance liquid chromatography; LC=long chain aminocaproic acid; HSA=human serum albumin; NHS=N-hydroxysuccinimide; PMT=photomultiplier tube; kU=kilo unit; mIU=milli-international unit; L=liter; mL=milliliter; min=minute; dPEG4-biotin=

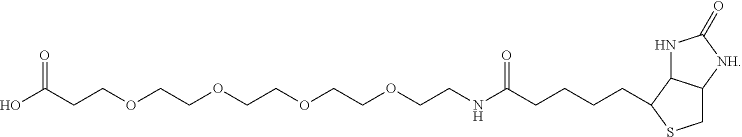 [SMILES: O=C(O)CCOCCOCCOCCOCCNC(=O)CCCCC1SCC2NC(=O)NC21]

Preparation of melittin peptide: The 26-amino acid melittin peptide (Gly-Ile-Gly-Ala-Val-Leu-Lys-Val-Leu-Thr-Thr-Gly-Leu-Pro-Ala-Leu-Ile-SerTrp-Ile-Lys-Arg-Lys-Arg-Gln-Gln) (SEQ ID NO: 1) was prepared by solid-phase peptide synthesis on a SONATA® peptide synthesizer (Protein Technologies, Inc., Tucson Ariz.) using Fmoc (N-fluorenylmethyloxycarbonyl) chemistry. In the solid-phase peptide synthesis, the peptide was assembled by coupling one amino acid in each cycle starting with the C-terminal amino acid attached to the resin. The peptides employed were N-α-Fmoc-glycine, N-α-Fmoc-L-isoleucine, N-α-Fmoc-L-alanine, N-α-Fmoc-L-valine, N-α-Fmoc-L-leucine, N-α-Fmoc-N-ε-tert-Boc-L-lysine, N-α-Fmoc-O-tert-butyl-L-threonine, N-α-Fmoc-L-proline, N-α-Fmoc-O-t-butyl-L-serine, N-α-Fmoc-N-in-tert-Boc-L-tryptophan, N-α-Fmoc-NG-(2,2,4,6,7-pentamethyldihydrobenzofuran-5-sulfonyl)-L-arginine and N-α-Fmoc-γ-trityl-L-glutamine. At the end of the solid-phase peptide synthesis, the peptide connected to the resin at the C-terminus contained lysines with an ε-amino group protected by Boc, threonine and serine with hydroxyls protected as tert-butyl ethers, tryptophan with the indole nitrogen protected by Boc, arginine with the guanidyl group protected by a 2,2,4,6,7-pentamethyldihydrobenzofuran-5-sulfonyl group and glutamine with the ε-amide nitrogen protected with a trityl group.

Preparation of Biotin-Melittin Peptide Conjugates: Biotinylation of the N-terminal amino group of the melittin peptide was carried out by treatment of the Fmoc-deprotected peptide resin with Biotin-LC-NHS (Critical Reagents Manufacturing, Siemens Healthcare Diagnostics, Los Angeles, Calif.) or Biotin-dPEG4-NHS (Quanta BioDesign LTD, Powell Ohio) in DMF with diisopropylethylamine. The free primary amino group of the N-terminal glycine of peptide-resin was reacted with excess of Biotin-dPEG4-NHS ester or Biotin-LC-NHS in the presence of DMF and diisopropylethyl amine. The excess reagents were washed away and then the peptide was cleaved from the resin using a cleavage cocktail containing phenol, trifluoroacetic acid, thioanisole and ethanedithiol. The crude peptide was precipitated by addition of ether, lyophilized and then purified by preparative reversed-phase HPLC. The accuracy of the peptide sequence was confirmed by N-terminal sequencing by Edman degradation using a PROCISE® Protein Sequencing System (Applied Biosystems, Foster City Calif.).

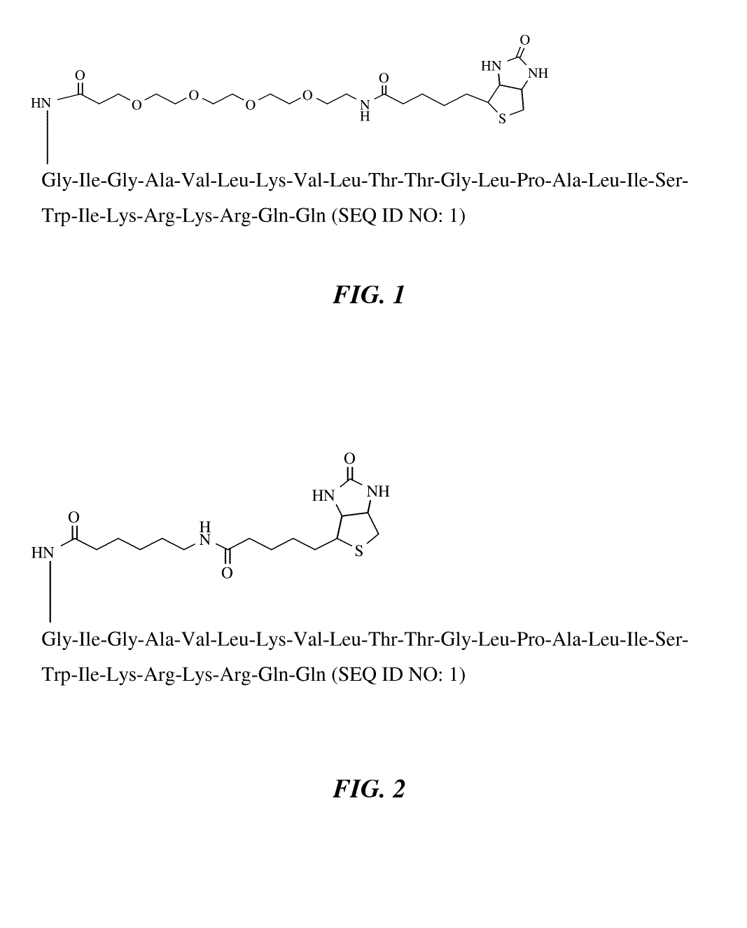

The Biotin-dPEG4-Melittin (see FIG. 1) was purified by preparative reversed-phase HPLC through a 250 mm×21.2 mm Phenomenex SYNERGI® C18 column (Phenomenex, Torrance Calif.) using a flow rate of 10 mL/min with absorbance detection at 214 nm. Solvent A was water with 0.05% trifluoroacetic acid. Solvent B was 80% acetonitrile, 20% water with 0.05% trifluoroacetic acid. The solvent gradient program consisted of 0% to 80% Solvent B in 60 min and then 80% to 100% Solvent B in 10 min. The compound that eluted with a retention time of about 51 min was collected and then lyophilized to produce the purified peptide. Electrospray-ion trap mass spectrometry gave a molecular weight of 3320 (calculated=3320 for C₁₅₂H₂₆₄O₃₈S). Analytical reversed-phase HPLC showed a purity of 100%. The Biotin-LC-Melittin (see FIG. 2) was prepared in a similar manner to that described above.

Preparation of MUXF allergen: MUXF-glycopeptides were obtained by enzymatic digestion of commercially available bromelain, a glycoprotein from pineapple stem. Bromelain was purified by dialysis and then digested with Pronase enzyme. The resulting mixture was further purified by column chromatography using a BIOGEL® P4 column (Bio-Rad Laboratories, Hercules Calif.), followed by filtration through a Millipore YM-3 membrane (3000 MW cut-off) (Millipore Corporation, Bedford Mass.). The mixture was then subjected to boronic acid gel chromatography followed by a final BIOGEL® P4 column purification. The MUXF-glycopeptides were identified by MALDI-TOF mass spectrometry and the presence of carbohydrate in the MUXF-glycopeptide was verified by a phenol-sulphuric acid method. The resulting purified MUXF-glycopeptides were further coupled to biotinylated HSA by EDC coupling to produce a conjugate that was demonstrated to be useful as an allergy diagnostic tool by means of an IMMULITE® 2000 3gAllergy™ assay (Siemens Healthcare Diagnostics Inc., Newark Del.).

Assay for IgE Analyte: The experiments were conducted on a Reagent Carousel of the IMMULITE® 2000/2500 3gAllergy assay (Siemens Healthcare Diagnostics Inc.). This assay system measures an immune response, in this case, the amount of immunoglobulin E (IgE), to specific allergen, in patient serum. Both the anti-IgE and the allergen are liquid and are stored in bar-coded vials that fit into the Allergen Wedge on the Reagent Carousel. Any number of biotinylated allergens can be used with the 3gAllergy system. The 3gAllergy assay on the IMMULITE® 2000/2500 is a 2-cycle assay. At the start of the first cycle patient serum containing specific IgE and biotinylated allergen (wherein the allergen was Biotin-LC-Melittin or Biotin-dPEG4-Melittin) from bar-coded test tubes were added simultaneously to a streptavidin-coated bead. The mixture was incubated for 30 minutes at 37° C. The patient's IgE antibody (Ab) recognizes and binds to the allergen. The IgE Ab/biotinylated allergen complex binds to the streptavidin-coated bead. Unbound material was removed through a wash cycle. At the start of the second cycle alkaline phosphate (enzyme) labeled anti-IgE from the Reagent Wedge was added to the reaction medium followed by a second 30 minute incubation at 37° C. After the second 30-minute incubation, the reaction tube was washed to remove any unbound alkaline phosphatase labeled anti-IgE. Substrate solution containing the chemiluminescent substrate PPD (4-methoxy-4-(3-phosphate-phenyl)-spiro-(1,2-dioxetane)-3,2′-adamantane) and TB enhancer (poly(vinylbenzyl-tri(n-butyl)phosphonium chloride)) was then added to the washed bead reagent and the light generated was measured by the PMT after a 5-minute incubation at 37° C. in a luminometer. The intensity of the light is proportional to the amount of IgE in the patient sample. The IMMULITE® 2000/2500 calibration method employs a stored master curve in conjunction with a two-point adjustment procedure. Units reported are kU/L or mIU/ml.

Results: Results were calculated using a point-to-point formula method, in which several standards are run. Each standard had a specific concentration and a corresponding signal. A master curve was generated when each standard was connected point-to-point by a straight line. Allergy level was reported in two ways: (a) concentration of IgE kU/L and (b) class (classes are based on concentration); two classifications exist (Standard and Extended). The following is an example of a standard classification: Class 0=<0.35 kU/L, Class 1=0.35−0.7 kU/L, Class 2=0.7−3.5 kU/L, Class 3=3.5−17.5 kU/L, Class 4=17.5−52.5 kU/L, Class 5=52.5−100 kU/L, and Class 6=>100 kU/L. The assay results for allergy patient samples obtained from the Technical University of Munich are summarized in Table 1 which represents a crosstable evaluation wherein N=number of samples.

The assay results provide the allergy levels according to classification of the patient samples resulting from the chemiluminescent immunoassay of IgE. The results show that the assay using the purified single species N-terminal biotinylated synthetic Melittin peptide in accordance with the principles described herein (“Test Method”) accurately detected IgE specific for Api m4 allergen when compared to the use of MUXF allergen in the 3gAllergy assay on the IMMULITE® 2000/2500 as a reference (“Ref Method”). The MUXF assay is an in-vitro allergy immunodiagnostic tool to address IgE reactivity to cross-reactive carbohydrate determinants (CCD) in patient sera. Clinically irrelevant immunoassay results, particularly with food allergens of plant origin, are known to occur in patient sera because of IgE reactivity to CCD. The MUXF assay detects the presence of a CCD structure (i.e., MUXF-glycopeptides) as an immunodiagnostic tool for detection of anti-CCD IgE reactivity. The IgE reactive CCD structure, commonly referred to as MUXF, is Manα1-6 (Xyl(β1-2) Manβ1-4GlcNAcβ1-4 (Fucα1-3) GlcNAc.

TABLE 1

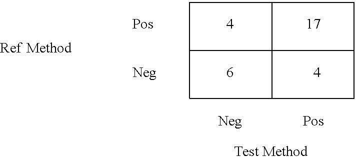

| N | 31 | |
|---|---|---|
| Total Agreement | 74.2% | 23/31 |
| Relative Sensitivity | 81.0% | 17/21 |
| Relative Specificity | 60.0% | 6/10 |
| Pos. Predictive Value | 81.0% | 17/21 |
| Neg. Predictive Value | 60.0% | 6/10 |

All publications and patent applications cited in this specification are herein incorporated by reference as if each individual publication or patent application were specifically and individually indicated to be incorporated by reference.

It should be understood that the above-described examples are merely illustrative of some of the many specific examples that represent the principles described herein. Clearly, those skilled in the art can readily devise numerous other arrangements without departing from the scope as defined by the following claims.

What is claimed is:

1. A reagent for determining in a sample the presence and/or amount of an IgE specific for a honey bee venom allergen, the reagent consisting of a conjugate of a small molecule linked to the terminal glycine amino acid of a synthetic 26 amino acid melittin peptide.

2. The reagent according to claim 1 wherein the small molecule is biotin.

3. The reagent according to claim 1 wherein the small molecule is linked to the terminal amine nitrogen of the peptide by a linking group comprising 2 to 100 atoms in a chain wherein the atoms in the chain are independently selected from the group consisting of carbon, oxygen, nitrogen, sulfur and phosphorus.

4. The reagent according to claim 1 wherein the small molecule is linked to the terminal amine nitrogen of the peptide by a linking group comprising repeating ethylene oxide units.

5. The reagent according to claim 4 wherein the number of repeating ethylene oxide units is four.

6. A method for determining in a sample the presence and/or amount of an IgE specific for a honey bee venom allergen, the method comprising:
(a) providing in combination in a medium:
(i) the sample, and
(ii) the reagent according to claim 1,
(b) subjecting the combination to conditions for binding of the IgE to the reagent to form a complex, and
(c) detecting the presence and/or amount of the complex, the amount of the complex being related to the presence and/or amount of the IgE in the sample.

7. The method according to claim 6 wherein the small molecule is biotin.

8. The method according to claim 6 wherein the honey bee allergen is Api m4 allergen.

9. A method for determining in a sample the presence and/or amount of an IgE specific for a honey bee venom allergen, the method comprising:
(a) providing in combination in a medium:
(i) the sample, and
(ii) a reagent consisting of a conjugate of a small molecule linked to the terminal glycine amino acid of a synthetic 26 amino acid melittin peptide,
(b) subjecting the combination to conditions for binding of the IgE to the reagent to form a complex, and
(c) detecting the presence and/or amount of the complex, the amount of the complex being related to the presence and/or amount of the IgE in the sample.

10. The method according to claim 9 wherein the small molecule is biotin.

11. The method according to claim 9 wherein the small molecule is linked to the terminal amine nitrogen of the peptide by a linking group comprising 2 to 100 atoms in a chain wherein the atoms in the chain are independently selected from the group consisting of carbon, oxygen, nitrogen, sulfur and phosphorus.

12. The method according to claim 9 wherein the combination further comprises a binding partner for the small molecule bound to a particle.

13. The method according to claim 9 wherein the conjugate is bound to a particle.

14. The method according to claim 9 wherein the complex is detected by adding to the medium an antibody for IgE wherein the antibody comprises a label.

15. The method according to claim 14 wherein the label is an enzyme, a fluorescent molecule, a chemiluminescent molecule, a radioisotope or a sensitizer.

16. A method for determining in a sample the presence and/or amount of an IgE specific for Api m4 allergen, the method comprising:
(a) providing in combination in a medium:
(i) the sample, and
(ii) a reagent consisting of a conjugate of a small molecule linked to the terminal glycine amino acid of a synthetic 26 amino acid melittin peptide by means of a linking group comprising repeating ethylene oxide units,
(b) subjecting the combination to conditions for binding of the IgE to the reagent to form a complex, and
(c) detecting the presence and/or amount of the complex, the amount of the complex being related to the presence and/or amount of the IgE in the sample.

17. The method according to claim 16 wherein the combination further comprises a binding partner for biotin bound to a particle.

18. The method according to claim 16 wherein the conjugate is bound to a particle.

19. The method according to claim 16 wherein the complex is detected by adding to the medium an antibody for IgE wherein the antibody comprises a label.

20. The method according to claim 19 wherein the label is an enzyme, a fluorescent molecule, a chemiluminescent molecule, a radioisotope or a sensitizer.


SEQUENCE LISTING (1 sequence)

SEQ ID NO: 1 - protein, 26 aa, Apis (various species)
  PEPTIDE (1)..(26)
GIGAVLKVLTTGLPALISWIKRKRQQ